(19) United States
(12) Patent Application Publication
(10) Pub. No.: US 2009/0299113 A1
(43) Pub. Date: Dec. 3, 2009

(54) PROCESS FOR PREPARING ALKANE LIQUIDS FOR USE IN IMMERSION LITHOGRAPHY

(75) Inventors: Douglas J. Adelman, Wilmington, DE (US); Hoang Vi Tran, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. duPont de Nemours and Company, Wilmington, DE (US)

(21) Appl. No.: 12/128,020

(22) Filed: May 28, 2008

(51) Int. Cl. C07C 5/02

(52) U.S. Cl. 585/264

(57) ABSTRACT

A method for purifying liquid alkanes, especially dicyclic alkanes, for use in immersion lithography is provided. The method produces alkanes having absorbance at 193 nm of ≦0.1/cm, and residue of ≦100 ppm. The liquid alkane compositions are useful as immersion liquids in photomicrolithography employed for production of electronic circuits.

### FIELD OF THE INVENTION

The present invention is directed to a process for preparing low absorbance and low residue alkane liquids for use as immersion liquids in photomicrolithographic production of electronic circuits.

### BACKGROUND

Methods suitable for the purification of alkanes have long been known in the art. Such methods include hydrogenation to remove unsaturated impurities, adsorbent beds, zone refining, distillation, concentrated acid wash and so forth.

Dumitrescu et al., Romanian Patent RO 101217, discloses the use of hydrogenation for removing unsaturated impurities from n-hexane.

Japanese Patent Application JP 03031304 A (abstract), discloses purification of hexanes by water washing, distillation, treatment in an adsorbent bed, followed by hydrogenation.

Teteruk et al. SO Neftepererabotka i Neftekhimiya (Moscow, Russian Federation) (1988), (7), 18-19 (abstract only), reports a comparison between hydrogenation and adsorption for the purification of hydrocarbons. The report concludes that adsorption is more effective.

Photolithographic methods have been employed for decades to fabricate electronic integrated circuits, and more recently, integrated optical circuit elements. One key enabling technology for fabricating ever-higher density integrated circuits has been the application of shorter and shorter wavelengths of exposure light, the smaller wavelengths permitting resolution of finer lines. Current technology employs ultraviolet (UV) wavelengths, generally below 250 nm, especially at 193 nm, in order to achieve the highest resolution possible in the present state of the art.

Recently it has been found that introduction of a high refractive index liquid in place of air between the last lens element and the photosensitive target enables the production of higher resolution images at 193 nm illumination. Switkes et. al., Proceedings of SPIE, Volume 5040, 699 (2003) discusses so-called immersion photolithography. Water has been the immersion liquid of choice in photolithography with a 193 nm light source.

Low absorbance of the immersion liquid is of great importance. For a given degree of light transmission to the photosensitive target surface, lower absorbance equates to greater working distance, which is of great practical value. Furthermore, lower absorbance results in less radiative heating of the fluid. Because refractive index is temperature dependent, a change in temperature in the liquid can cause blurring of the image.

Minimization of low volatility contaminants is also of great importance. Any residue remaining on the target surface after evaporation of immersion liquid is likely to have detrimental effects on the quality of the image formed thereupon.

Hydrocarbons, especially alkanes, are known to exhibit refractive indices higher than that of water. For example, replacement of water as an immersion liquid by bicyclohexyl, with a refractive index of 1.64, would reduce the effective wavelength of 193 nm light to 118 nm. However, to be of practical use, immersion liquids must also be transparent. The absorbance requirements suitable for practical use appear to be ever tightening. For example, Zhang et al., U.S. Published Patent Application 2005/0173682, describe immersion fluids characterized by absorbance of 5 cm⁻¹ whereas today, practical absorbance is thought to lie at ≦0.10 cm⁻¹.

Miyamatsu et al., WO2005/114711 (examples and claims only), disclose a process for preparing highly transparent alkanes by a combination of treatment with sulfuric acid and distillation.

French et al., WO2005/119371 discloses methods for purifying alkanes to achieve high transparency including hydrogenation and adsorbent treatment.

### SUMMARY OF THE INVENTION

One embodiment of the present invention is a process comprising distilling a composition comprising a liquid alkane to prepare a heart-cut distillate, contacting said distillate with hydrogen in the presence of a supported catalyst, under such conditions as to effect the hydrogenation of unsaturated species that may be present in said distillate to produce a hydrogenated alkane composition; and, filtering said hydrogenated alkane composition to produce a filtrate having an absorbance at 193 nm of 0.1 cm⁻¹ or less, and a residue of less than 1 ppm.

In a further embodiment thereof, an adsorption step is performed after said hydrogenation.

### BRIEF DESCRIPTION OF THE DRAWINGS

FIG. 1 illustrates a distillation apparatus according to embodiments of the present invention.

### DETAILED DESCRIPTION

For the purposes of the present invention, a liquid alkane composition is suitable for use in immersion lithography when the absorbance thereof at 193 nm is ≦0.1 cm⁻¹, the residue level is 1 ppm or less, and the presence of oxygen, water, and potential bubble forming impurities are minimized as described infra.

For the purposes of the present invention, the term “liquid alkane composition” refers to a composition comprising a preponderance of one or more liquid alkanes. By “preponderance”, as used herein, is meant at least 90% by volume, preferably at least 95% by volume, most preferably at least 98% by volume of one or more liquid alkanes, which composition can also include various impurities and contaminants. Some impurities have a more deleterious effect on the utility of a liquid alkane composition in immersion lithography that do others. Even up to levels of a few %, impurities such as normally gaseous alkanes, or that have particularly high relative vapor pressures (referred to herein as “volatiles.”) will have little effect on the utility of the liquid alkane composition because they do not reduce absorbance. Those impurities include methane, ethane, propane, butane. Other impurities, such as olefins or ketones, for example, will have a deleterious effect on absorbance at concentrations at parts per million level.

It is believed that under some conditions of use in immersion lithography, microscopic bubbles that scatter light might form when there is an excess of volatiles in the immersion liquid. Variable volatiles concentration at overall concentration of about 0.1% by volume can cause an undesirable variability in refractive index from time to time. It is therefor of considerable value to reduce volatiles concentration to less than 0.1% by volume, preferably less than 0.01% by volume,

Impurities can also include dissolved species that are normally solid or waxy at room temperature. Solid or waxy species can leave undesirable residues on the surfaces of silicon wafers that have been subject to immersion lithography using liquid alkane compositions. Impurities can further include unsaturated species, such as ketones and olefins, that are highly absorbing at 193 nm, the particular wavelength of interest herein.

The operability of the process disclosed herein is not diminished if the liquid alkane composition comprises a plurality of liquid alkanes intermixed with one another, provided each liquid alkane is suitable for the practice of the invention.

Polycyclic alkanes, particularly dicyclic alkanes, are generally preferred for their combination of high refractive index, low vapor pressure, low residue, and low absorbance at 193 nm that they display after purification in accord with the present invention. Particularly preferred are bicyclohexyl, exo-tetrahydrodicyclopentadiene, and decahydronaphthalene. More preferred are bicyclohexyl and exo-tetrahydrodicyclopentadiene. Most preferred is bicyclohexyl (BCH).

The application of alkanes to immersion lithography places unprecedented demands upon the purity and stability of the alkanes so intended. Immersion lithography demands of the immersion liquid extremely high transparency, extremely low light scattering, extremely stable refractive index, extremely low surface contamination, all in combination with refractive index higher than 1.60. Alkane compositions suitable for this use—that is that met the requirements of the application—did not exist when immersion lithography was introduced by Switkes, op. cit., and others in 2003. As the technology has advanced, the requirements have tightened. Current requirements for utility in immersion lithography include absorbance at 193 nm≦0.10/cm, and residue of ≦100 ppm, preferably ≦1 ppm along with a refractive index above 1.60. The alkane compositions prepared according to the process meet those requirements for utility in immersion lithography.

As used herein, the term “absorbance” refers to the thickness normalized decrease in the intensity of light having a wavelength of 193 nm that has been transmitted through a specimen, as described by the equation,

α=[ln(lₒ/l)]/l

where α is absorbance, l₀ is the intensity of incident light, l, the intensity of transmitted light, and t is the optical path length through the specimen, in centimeters.

Alkane compositions prepared according to the process disclosed herein have an absorbance at 193 nm of ≦0.10/cm. For the purposes of the present invention, it is satisfactory to determine absorbance with a Varian Cary 5 UV/Vis/NIR spectrometer at 193 nm in glass cuvettes loaded under nitrogen.

As used herein, the term “residue” refers to the amount of solid material remaining on a silicon surface after a droplet of alkane containing that residue is evaporated from the silicon surface. The residue is expressed in parts per million (ppm) on a volume basis. The method for determination of residue is described in detail infra. The alkane compositions prepared by the process disclosed herein are characterized by a residue level of ≦100 ppm, preferably ≦1 ppm.

Every chemical in the real world has some level of contamination from starting materials, side reactions, handling vessels, the atmosphere, UV degradation, and so forth. Even very pure compounds, such as chromatographic calibration standards, would be expected to have some degree of contamination. Consequently, it can never be known what the intrinsic absorbance of a chemical actually is, because there always remains the possibility that a very small amount of a highly absorbing species could be present. It can only be known after the removal of that species that the intrinsic absorbance of the chemical in question is lower than the previous lowest value. As purity levels get higher, proving the existence of, identification of, and removal of a contaminating species becomes increasingly problematical. It is equally problematical to increase purity by any process while avoiding contamination by the purification process itself.

Liquid alkanes suitable for use as starting materials in the process are well known and widely available commercially, in a wide range of purities. Suitable liquid alkane starting materials include linear, cyclic, and multi-cyclic alkanes. Multi-cyclic alkanes are preferred. Preferred multi-cyclic alkanes are bicyclohexyl, exo-tetrahydrodicyclopentadiene, and decahydronaphthalene. More preferred are bicyclohexyl and exo-tetrahydrodicyclopentadiene. Most preferred is bicyclohexyl.

The liquid alkane starting material is subject to fractional distillation followed by hydrogenation. It has been found that when the liquid starting material is first subject to hydrogenation followed by distillation, the result obtained is not suitable. The sequence of the unit operations performed is critical to the successful outcome: preparing a liquid alkane that is suitable for use in immersion lithography at 193 nm.

Distillation is a well-known process. A variety of configurations for effecting distillation of liquids are known. See, for example, Perry's Chemical Engineer's Handbook, 8^{th} ed., D. W. Green, editor, Chapter 12:Distillation, McGraw Hill (2007). Both batchwise and continuous distillation are suitable for the processes disclosed herein. Both single column and multi-column stills are suitable. The preferred process and preferred apparatus will be determined by the specific requisites of the application of the process. The discussion infra is largely directed to batchwise distillation, as are the specific embodiments of the process that are herein included. However, it is to be understood that the discussion is readily adapted to continuous distillation processes by an ordinary practitioner of the art. Vacuum distillation is suitable for the present invention.

Regardless of the specific design of the distillation apparatus and conditions employed, a suitable distillation process produces at least two fractions, namely, a product fraction and a bottoms fraction. In one embodiment, the distillation process further produces a volatiles fraction, with the product fraction following distillation of the volatiles fraction. These are terms in common use in the art of distillation.

As used herein, the term “volatiles fraction” refers to a distillate fraction that is characterized by a concentration of volatile contaminants in the liquid alkane distillate that is higher than that in the liquid alkane starting material. A volatile contaminant is one that has a higher vapor pressure than that of the liquid alkane in which it is a contaminant.

As used herein, the term “bottoms fraction” refers to that fraction of the liquid alkane starting material that remains in the distillation vessel at the conclusion of a batchwise distillation according to the process. The bottoms fraction has a higher concentration of lower vapor pressure residues in the liquid alkane than was present in the liquid alkane starting material. Often the bottoms fraction comprises a waxy substance that can leave a deposit on the imaged surface during the lithographic process.

As used herein, the term “product fraction” refers to that distillate fraction that is characterized by a concentration of low vapor pressure residues that is lower than that in the liquid alkane starting material. In the case where a separate volatiles fraction is distilled off before the product fraction, the product fraction is characterized in that the concentrations of both volatile contaminants and low vapor pressure residues are lower than those in the liquid alkane starting material. It is the product fraction that is employed in the hydrogenation step of the process. The volatile fraction, if any, and the bottoms fraction may be discarded or recycled for further distillation.

The distillation process may include a plurality of fractions that each individually conform to the definition of the term “product fraction.” The process is not limited by the number of product fractions that are produced during the distillation, nor does the process require that every such product fraction be employed in the hydrogenation; only that the hydrogenation step be performed using at least one such product fraction.

While there can be a plurality of product fractions, they are not necessarily of equal purity. In some embodiments of the process the product fraction may be subdivided into multiple fractions, and only the purest employed in hydrogenation.

Multiple fractions corresponding to the volatiles fraction may be produced as well. That in no way limits the operability of the present process.

The process is not limited by the number of times distillation is performed on a given quantity of liquid alkane prior to hydrogenation. While it is preferred to control distillation conditions so that only one distillation is required, the process encompasses a distillation step prior to hydrogenation that may in fact comprise a plurality of discrete distillations.

The distillation conditions represent a trade-off of several competing factors. It is desirable to exclude oxygen from the system to avoid any oxidation of the product. This is best accomplished by distilling at atmospheric pressure above the boiling liquid. However, it is found in the practice of the invention that at atmospheric pressure the liquid boiling point of 227° C. in the case of BCH is high enough to cause some degradation of the BCH during distillation. To reduce the boiling point, it is necessary to reduce the head space pressure, preferably to ca. 2 torr, where the boiling point is reduced to ca. 118° C. When the pressure is reduced, precaution must be taken to minimize leakage of outside air because oxygen can degrade the absorbance of the liquid alkane compositions. In common practice, vacuum grease is used to achieve vacuum tightness. However, it has been found in the practice of the invention that employing grease in the process can result in contamination of the distillate. So, as an alternative to grease, in one embodiment, the parts are assembled using Teflon® sleeves instead of grease. In another embodiment, grease is employed but applied in minimal quantities. In still another embodiment, the grease is replaced with Teflon®sleeves only in that portion of the apparatus that is wetted by fluid which is above the product removal point.

Satisfactory vacuum tightness is determined by charging the distillation flask with the liquid alkane starting material, and before heating it, connecting the system to a mechanical vacuum pump. The system is subject to pumping for ca. 15 minutes after which the pressure is determined to be 2 torr or less. A pressure greater than 2 torr is considered unsatisfactory leakage, and the distillation is aborted until the leak is repaired.

According to the present invention, distillation of BCH is satisfactorily conducted at a temperature in the range of 118-130° C., and a pressure in the range of 2-20 torr. 118-125° C. and 2-5 torr are preferred.

The product fraction obtained from the distillation step as described supra, is subject to hydrogenation. Unsaturated species such as olefins, ketones, aromatics and so forth, tend to be highly absorbing at 193 nm, and are common contaminants in alkanes. The purpose of hydrogenation is to reduce the concentrations of those species to low levels. Preferably, hydrogenation is conducted to a point at which unsaturated species are no longer detectable by gas chromatography/mass spectroscopy; that is, to a level below about 10 parts per million. Some, but not all, of the saturated analogs to the unsaturated contaminants originally present that remain after hydrogenation are much less absorbing, and therefore are of less concern as contaminants that cause absorption.

The duration of the hydrogenation step will depend upon the level of contamination by unsaturated species of the product fraction of the distillate. In the most preferred embodiment, wherein the alkane is bicyclohexyl, it is found in the practice of the invention that the most significant unsaturated species present in the product fraction from the distillation is biphenyl.

Hydrogenation is advantageously conducted in the presence of a catalyst. Suitable catalysts include, but are not limited to, ruthenium, palladium, platinum, Raney nickel, rhenium, and rhodium. Only inhomogeneous catalysis is suitable for the practice of the present invention since it is desirable to remove all catalyst after hydrogenation to keep light scattering as low as possible. In general, it is expected that the best choice of catalyst will vary with the particular alkane to be purified and with the contaminants to be hydrogenated, and the choice can readily be made by one skilled in the art. It has been found in the practice of the invention that rhodium on carbon is well-suited for use in the hydrogenation of bicyclohexyl compositions, and palladium on carbon is well-suited for use in the hydrogenation of exo-tetrahydrodicyclopentadiene compositions.

Hydrogenation can be conducted in the temperature range of 70-200° C., preferably 80-120° C., and at pressures in the range of 100-1000 psi (0.69-6.9 MPa) of hydrogen, preferably 500 to 1000 psi (3.5-6.9 MPa), for a period of 1 to 48 hours, preferably at least 5 hours. Hydrogenation is suitably carried out in a grease-free corrosion resistant autoclave that has been carefully cleaned prior to use. 100° C. is preferred for hydrogenation of bicyclohexyl and exo-tetrahydrodicyclopentadiene compositions. Bicyclohexyl hydrogenation is preferably accomplished using rhodium on carbon as a catalyst.

To prevent light scattering from catalyst residues, the hydrogenated alkane composition can be filtered. Filtration of the hydrogenated product can be accomplished using a commercially available sub-micron filter. Preferably, sintered metal filters designed to capture sub-micron particles are used to prevent contamination of the fluid by soluble components present in plastic filters. Alternatively, filtration can be accomplished by using an adsorbent column. In another alternative, the catalyst can be removed by centrifugation and decanting.

The amount of residue is subject to increase the more the refined product is handled. Distillation has been found to be quite effective for reducing the solid residue levels to ≦1 ppm. However, in the practice of the invention at the laboratory scale, the distillate undergoes several transfer steps in laboratory equipment and any one or combination thereof can, and sometimes does, recontaminate the distillate with solids that are then present as residue. While it is expected that in a dedicated commercial operation residue levels will be below 1 ppm, in the process as herein practiced, it is found that levels between 1 ppm and 100 ppm will sometimes be produced.

The most appropriate particulars for a given set of circumstances of the distillation apparatus and conditions, and the relative size of the product fraction, will depend upon the target absorbance and residue levels as well as the absorbance and residue levels of the starting materials. In a preferred embodiment, the alkane composition used as the starting material in the process comprises at least 95%, more preferably at least 98% alkane and can be a mixture of liquid alkanes. As a practical matter, the starting material in the process may itself be the product of other purification treatments in order to achieve the level of purity desired for the starting material of the process. Such purification treatments may include distillation, hydrogenation, filtering, zone-refining, treatment with adsorbent beds, concentrated acid wash, and other such treatments as are commonly employed in the art for purifying alkanes

The process of the present invention consists essentially of distillation of a liquid alkane composition to produce a product fraction, followed by hydrogenation of said product fraction to produce a hydrogenated product fraction, and filtration of said hydrogenated product fraction to produce a liquid alkane composition characterized by an absorbance at 193 nm of ≦0.1/cm and a residue of ≦100 ppm, preferably ≦1 ppm.

### EXAMPLES

### Distillation

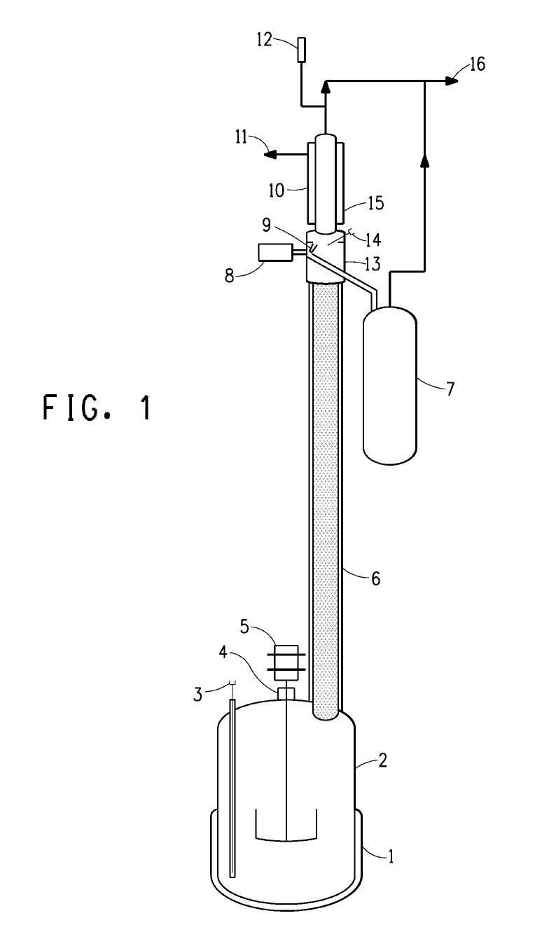

FIG. 1 depicts the distillation apparatus employed in the specific embodiments, infra. An external electric heating mantle, 1, was used to heat a stirred 22 liter glass round bottom flask, 2. The flask was fitted with a thermocouple well and thermocouple to provide temperature control. through a 29/42 ground glass joint, 3, lined with Teflon®. The upper half was thermally insulated. A stirring motor, 5, was attached to the internal stirrer via a Teflon® stirrer bearing, 4, inserted in a 45/50 ground glass joint. Attached to the reactor via a 55/50 ground glass joint was a 2″ ID vacuum jacketed glass column, 6, packed with ¼″ stainless steel Propak. The packed length was 40″. An 18 liter stainless steel receiver with all the gaskets of which were made of Teflon® was connected to the condensate line through a 28/5 ball and socket joint fitted with a Viton® o-ring.

A vacuum jacketed distillation head, 13, was connected to the top of the column, 6, with a 55/50 ground glass joint lined with a Teflon® sleeve on Within the still head was a swinging-bucket valve, 9, whose position was controlled by an electromagnet, 8, that controlled the reflux ratio during distillation. There was a thermocouple well and thermocouple, 14, in the still head to read the distillate temperature. An Allihn condenser, 10, was connected to the still head, 13, by a Teflon® lined 55/50 ground glass joint. The condenser, 10, was 2″ ID×17″ long and was cooled by water at 15° C. at the inlet, 15. The condenser coolant stream exited at 11. Vacuum was provided at the exit of the condenser by mechanical pump through a dry ice trap, 16. There was a pressure transducer, 12, located on the condenser vapor exit line.

The unit was degreased originally by boiling acetone up through the unit. All joints were wiped down with dichloromethane.

Finally, 2 BCH boil-outs were done to wash out any other contaminates.

All ground glass joints are sealed with Teflon sleeves. No other fluids are processed in the unit. No clean-outs are done between batches to prevent contamination of the system by the clean-out fluid. The distillation heel was pulled out and then the unit was held under a nitrogen purge prior to the next distillation.

### Hydrogenation

Hydrogenation was performed in a 5 gallon stainless steel stirred autoclave (Autoclave Engineers, Inc) provided with an air driven agitator shaft with 3 impellers affixed thereto at vertical intervals to provide stirring from top to bottom. The autoclave was electrically heated using an external band heater. Cooling was achieved using circulating water internal coils.

Prior to use, the interior of the autoclave was subjected to washing using three 5 liter aliquots of 10% sulfuric acid, stirring for two hours for each washing. The acid wash was followed by rinsing for two hours using three 5 liter aliquots of deionized water. After the water washing, three similar washings were performed using three 5 liter aliquots of reagent grade methanol, followed in turn by three 5 liter aliquots of reagent grade acetone, and three 5 liter aliquots of Vertrel® XF (DuPont) fluorinated solvent. Following the last washing, the reactor was wiped with clean towels followed by nitrogen purging.

The autoclave was equipped with an agitator driven by a MagnaDrive (MagnaDrive Corporation, Bellevue, Wash.) adjustable speed drive. Prior to hydrogenation, the drive assembly was disassembled, and all fittings removed. All parts were subject to steam cleaning, followed by rinsing with Vertrel® XF. The drive was then reassembled using all new Purebon® bearings. Reactor head fittings were inspected for any sign of corrosion and were replaced as necessary.

Also prior to hydrogenation, all fittings and lines were disassembled and steam cleaned followed by rinsing with Vertrel® XF, and were replaced as necessary.

All the parts were then reassembled, the reactor sealed and leak tested to 850 psi with nitrogen.

### Characterization

Absorbance was measured with a Varian Cary 5 UV/Vis/NIR spectrometer at 193 nm in glass cuvettes having optical path lengths that were varied depending upon the absorbance of the set of samples being tested in order to get a meaningful result. Cuvettes were loaded under nitrogen.

Catalysts employed were 5% Ruthenium on Carbon: (Aldrich catalog #20,618-0), 5% Platinum on Alumina: (Acros catalog #195260100), 5% Palladium on Carbon (Aldrich catalog #33,012-4), 60 wt-% Nickel 60 on Kieselguhr (Aldrich catalog #20,878-7), and 5% Rhodium catalyst on carbon (Aldrich)

Liquid samples were stored and handled using TraceClean™ bottles supplied by VWR International, Inc., West Chester, Pa. 19380. Overall, liquid handling was done as much as possible under nitrogen using clean, grease free equipment.

Residue determination was accomplished as follows: A silicon wafer was cleaned using acetone and Vertrel® XF (DuPont). A 1-2 microliter droplet was placed on the surface of the clean wafer, creating a circular footprint of 2-5 mm diameter. The droplet so formed was evaporated at 90° C. on a hotplate until the diameter was approximately 1 mm. To determine the volume fraction of residues in the droplet, the 1 mm diameter droplet was first subject to imaging using an Olympus Lext OLS 3100 confocal microscope (Olympus Surgical and Industrial America Inc, Micro-imaging Division, Orangeburg, N.Y.). The droplet was then allowed to evaporate, leaving behind one or more residues. Each residue thus formed was similarly imaged. The volume of the original droplet and each residue was determined by image analysis using Lext OLS software that accompanied the microscope. The sum of the volumes of the residues was compared to the volume of the original droplet to determine the volume fraction of residue.

Absorbance of as-received bicyclohexyl (Solutia 99.6%.) was >300 cm⁻¹ at 193 nm and the volume residue was 20 ppm. Absorbance of exo-tetrahydrodicyclopentadiene (Dixie Chemical −99+ %) was 16 cm⁻¹.

### Comparative Example A

10.314 kg of bicyclohexyl was charged to a 5 gal stainless steel autoclave, to which 23 g of 5% Ru on carbon was added. The autoclave was sealed, heated to 200° C. and pressurized to 800 psi (5.5 Mpa) with hydrogen. The conditions were maintained for 8 hours, followed by cool-down and discharge of the hydrogenated product. The thus hydrogenated bicycylohexyl was found to have an absorbance at 193.4 nm of 0.160/cm, and a residue of 1.5 ppm. The hydrogenated fluid was pushed out of the autoclave through 2 sintered metal filters to 4 liter TraceClean™ bottles.

10.089 kg of the thus hydrogenated bicyclohexyl (BCH) was added to a 22 liter glass flask that was still wetted with fluid from a previous BCH distillation. No part of the system was cleaned out between distillations to prevent contamination by the clean-out fluid. The hydrogenated BCH was charged in the presence of air but then was outgassed under vacuum with a nitrogen purge for 10 minutes. Nitrogen purge was stopped and the vacuum of the system was monitored. If the pressure was not 2-5 mm Hg within 20 minutes, the ground joints and the stirrer bearing were checked for leaks. After stable vacuum was achieved, the fluid was heated and distilled up, through a 40″ long, 2″ inner diameter vacuum jacketed glass distillation column packed with ¼″ stainless steel mesh pieces (Propak Systems, Ltd.). The BCH was refluxed at a reflux ratio of 2 at 130° C. and 20 Torr while the highest volatility fraction was distilled off and collected in a 2 liter round bottom flask, wetted by BCH from previous distillations. When 4.7% by weight of the original charge had been distilled over, a volatiles fraction was removed. The reflux ratio was readjusted to 1, and the product fraction was then distilled. When 89.9% of the original charge had been distilled over, the distillation was stopped. The thus distilled product fraction had an absorbance at 193.4 nm of 0.62+/−0.12/cm. Residue was less than 0.6 ppm.

### Example 1

The same procedures for the distillation and hydrogenation described in Comparative Example A were employed, but the unit processes were operated in opposite sequence, distillation before hydrogenation. To a 22 liter glass flask was added 11.564 kg of BCH. The BCH was distilled in the column of Comparative Example A The BCH was held at 130° C. and 20 mm Hg while the highest volatility fractions were distilled with a reflux ratio of 3. When 6.7% of the original charge had been distilled, a volatiles fraction was removed. The product fraction was then distilled at a reflux ratio of 1, still at 130° C. and 22 torr. When 95.7% of the original charge had been distilled, the distillation was stopped. GC/MS analysis of the distillate showed traces of volatiles but no sign of higher boiling residues. Residue analysis showed less than 100 ppb residue.

10.134 kg of the distillate so produced was charged to the Hastelloy® autoclave and subject to hydrogenation as described in Comparative Example A except that herein 22 g of Ru on carbon was used. Absorbance of the thus hydrogenated BCH at 193.4 nm was 0.030+/−0.004/cm with 20 ppm residue. GC/MS revealed ca. 4% of volatiles.

### Example 2

12.939 kg of BCH was distilled in the manner of Example 1 except that the temperature was 118° C. and the pressure 2.9 torr. The volatiles fraction was 9.4%. The product fraction was concluded after distillation of 61.8% of the original charge. GC/FID analysis of the thus distilled BCH showed a trace of volatile contamination but no higher boiling residues. Residue was less than 2 ppm.

Hydrogenation of the BCH distillate was accomplished in the manner of Comparative Example B except that the temperature was 100° C. and the catalyst was 25 g of 5% Rh on carbon. Absorbance of the thus hydrogenated BCH at 193.4 nm was 0.032+/−0.009/cm. Residue was less than 0.100 ppm. GC with flame ionization detection showed no increase in the content of volatile contaminants.

### Examples 3-5

The methods and procedures of Example 1 were followed.

The quantities, conditions and results are shown in Table 1.

### Comparative Examples B-D

The methods and procedures of Comparative Example A were followed. The quantities, conditions and results are shown in Table 2.

### Comparative Example E

The methods and conditions of Example 1 were followed but the hydrogenation conditions employing the catalyst of 5% Ru on carbon at 100° C. were insufficiently strong to effect complete hydrogenation of unsaturated absorbing species in the time allotted for the reaction. Example 1, in contrast, shows that the same catalyst at 200° C. results in quite effective hydrogenation. Similarly, Examples 2-5 show that 5% Rh on carbon permits hydrogenation at lower temperatures while still providing a high degree of hydrogenation. Reaction parameters and results are shown in Table 1.

TABLE 1

| Distillation | | | | | Hydrogenation | | | | |
|---|---|---|---|---|---|---|---|---|---|
| | | Volatiles | Bottoms | | | | | | Final |
| | BCH | Fraction | Fraction | BCH | BCH | | Catalyst | Autoclave | Product |
| | Charge | (approx. | (approx. | Product | Charge | | Charge | Temperature | Absorbance |
| Example # | (g) | wt-%) | wt-%) | Cut (g) | (g) | Catalyst | (g) | (° C.) | (/cm) |
| 1 | 11564 | 6.7 | 4.3 | 10387 | 10134 | Ru/C | 22 | 200 | 0.030 |
| 2 | 12939 | 9.4 | 38.2 | 6781 | 6638 | Rh/C | 25 | 100 | 0.032 |
| 3 | 14341 | 12.1 | 16.5 | 10241 | 10164 | Rh/C | 35 | 100 | 0.040 |
| 4 | 14402 | 11.8 | 10.5 | 11168 | 11056 | Rh/C | 45 | 125 | 0.031 |
| 5 | 13719 | 9.8 | 4.4 | 11755 | 11726 | Rh/C | 25 | 70 | 0.040 |
| Comp. Ex. E | 13234 | 6.5 | 10 | 11054 | 10914 | Ru/C | 23 | 100 | >0.1 |

TABLE 2

| | | | | | Distillation | | | |
|---|---|---|---|---|---|---|---|---|
| Hydrogenation | | | | | | Volatiles | Bottoms | Final |
| | BCH | | Catalyst | Autoclave | BCH | Fraction | Fraction | Product |
| | Charge | | Charge | Temperature | Charge | (approx. | (approx. | Absorbance |
| Example # | (g) | Catalyst | (g) | (° C.) | (g) | wt-%) | wt-%) | (/cm) |
| Comp Ex A | 10314 | Ru/C | 23 | 200 | 10089 | 4.7 | 11.1 | 0.62 |
| Comp Ex B | 11530 | Rh/C | 25 | 125 | 8971 | 12.6 | 21 | >1.0 |
| Comp Ex C | 11672 | Ru/C | 25 | 200 | 11329 | 11.8 | 17.1 | 0.59 |
| Comp Ex D | 11310 | Ru/C | 25 | 200 | 9297 | 15.2 | 13.3 | 0.51 |

What we claim is:

1. A process consisting essentially of subjecting a liquid alkane composition to fractional distillation under vacuum to produce a product fraction, subjecting said product fraction to hydrogenation in the presence of an inhomogeneous catalyst to produce a hydrogenated product fraction, said hydrogenation being conducted at a temperature in the range of 70-200° C., and subjecting said hydrogenated product fraction to filtration producing a filtrate.

2. The process of claim 1 wherein the liquid alkane composition comprises a polycyclic alkane.

3. The process of claim 2 wherein the polycyclic alkane is bicyclohexyl, exo-tetrahydrodicyclopentadiene, or decahydronaphthalene.

4. The process of claim 3 wherein the polycyclic alkane is bicyclohexyl, or exo-tetrahydrodicyclopentadiene.

5. The process of claim 4 wherein the polycyclic alkane is bicyclohexyl.

6. The process of claim 1 wherein the catalyst is an inhomogeneous catalyst selected from the group consisting of ruthenium, palladium, platinum, raney nickel, rhenium, and rhodium.

7. The process of claim 4 wherein the catalyst is ruthenium on carbon, palladium on carbon, or rhodium on carbon.

8. The process of claim 5 wherein the catalyst is rhodium on carbon

9. The process of claim 1 wherein the catalyst is rhodium on carbon, and the hydrogenation is conducted at a temperature in the range of 80-120° C., and the liquid alkane composition comprises bicyclohexyl.

10. The process of claim 1 wherein the catalyst filtration step hydrogenated product fraction filtration is conducted using a sintered metal filter or an adsorbent bed.

11. The process of claim 10 wherein the adsorbent is silica.

12. The process of claim 1 wherein the liquid alkane composition comprises at least 98% by weight of a liquid alkane.
